(19) United States
(12) Patent Application Publication
(10) Pub. No.: US 2026/0183451 A1
(43) Pub. Date: Jul. 2, 2026

(54) COMPOSITE MATRIX USEFUL FOR PROMOTING INNERVATION, OSTEOGENESIS AND ANGIOGENESIS

(71) Applicants: INSTITUT POLYTECHNIQUE DE BORDEAUX, TALENCE (FR); UNIVERSITE DE BORDEAUX, BORDEAUX (FR); CENTRE NATIONAL DE LA RECHERCHE SCIENTIFIQUE, PARIS (FR); INSTITUT NATIONAL DE LA SANTE ET DE LA RECHERCHE MEDICALE, PARIS (FR)

(72) Inventors: BERTRAND GARBAY, MIOS (FR); SÉBASTIEN LECOMMANDOUX, CANEJAN (FR); ELISABETH GARANGER, TALENCE (FR); BRUNO PAIVA DOS SANTOS, MACAU (FR); NADIA MAHMOUDI, TALENCE (FR); HUGO CAMALHAO LOPES DE OLIVEIRA, MACAU (FR); JOËLLE AMEDEE, PESSAC (FR)

(21) Appl. No.: 18/858,797

(22) PCT Filed: Apr. 21, 2023
(86) PCT No.: PCT/EP2023/060524

§ 371 Date: Oct. 22, 2024

(30) Foreign Application Priority Data
Apr. 22, 2022 (FR) FR2203767

(51) Int. Cl. A61L 27/46; A61L 27/22; A61L 27/52

(52) U.S. Cl. CPC A61L 27/46; A61L 27/227; A61L 27/52; A61L 2400/06; A61L 2430/02

(57) ABSTRACT

The invention relates so a composite material useful for promoting innervation, osteogenesis and angiogenesis.

### TECHNICAL FIELD

The present invention relates to a composite matrix useful for promoting innervation, osteogenesis and angiogenesis.

### TECHNICAL BACKGROUND

The inventors' team (Silva et al, Cell Death and Disease, 2017 Dec. 13; 8(12):3209; Leroux et al, Cell Commun Signal. 2020 Oct. 19; 18(1):162) demonstrated, using co-culture models of sensory neurons, mesenchymal cells and endothelial cells, the impact of communication between these three cell types and the importance of neurovascular dialogue on osteogenesis. Building on these observations, this team developed an elastin peptide-based hydrogel able to stimulate recruitment of nerve fibers (Paiva dos Santos et al., Acta Biomaterialia, 2019 November; 99:154-167; PCT/EP2019/055075), in particular sensory neurons, and to accommodate other cell types. With this in mind, the inventors sought to improve various properties of said hydrogel, in particular in terms of cell colonization homogeneity and increased biodegradability of the material, and propose a biocompatible, injectable composite matrix able to stimulate innervation, tissue vascularization and to exhibit osteogenic properties for application in bone engineering.

### SUMMARY OF THE INVENTION

The inventors developed a new composite material comprising an organic phase and a mineral phase. This material is biocompatible, injectable, able to stimulate innervation and tissue vascularization, and to exhibit osteogenic properties for use in bone engineering. The invention relates more particularly to a composite matrix associating an organic phase functionalized by bioactive peptides and a mineral phase comprising calcium phosphate. The composite matrix according to the invention is able to recruit sensory neurons and to host osteoforming and endothelial cells.

### DETAILED DESCRIPTION

The composite matrix according to the invention is characterized in that it comprises an organic phase comprising an elastin-like peptide, at least one bioactive peptide and a mineral phase comprising calcium phosphate.

### 1. Elastin-Like Peptide

The first component of the composite matrix according to the invention is an elastin-like peptide (or ELP for Elastin-Like Polypeptide) comprising at least one methionine residue alkenylated before formation of the composite matrix. This type of peptide, the process for its manufacture by genetic engineering, and its purification are known to the person skilled in the art, who can refer in particular to the international application WO2017021334 and the articles Petitdemange et al. (Biomacromolecules. 2017 Feb. 13; 18(2):544-550) and Petitdemange et al. (Bioconjug Chem. 2017 May 17; 28(5):1403-1412). The skilled person can also refer to the examples presented below relating the production of the ELP designated ELPM80.

In the context of the present invention, the term “alkenylated methionine residue” means that the side chain of the methionine residue is covalently bonded to a moiety comprising an alkene group, i.e. comprising at least one double bond between two carbon atoms. Preferably, the term “alkene group” refers to the presence of a —CH═CH2 group in the moiety linked to the methionine residue. According to a particular embodiment, the methionine moiety is linked to the moiety of formula (I):

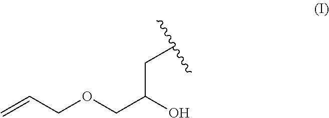 [SMILES: *CC(O)COCC=C]

According to one embodiment, the synthesis of an alkenylated ELP by means of the moiety of formula (I) can be performed by chemoselective thioalkylation at methionine side chains using an allyl glycidyl ether according to the procedure described in Petitdemange et al. (Bioconjug Chem. 2017 May 17; 28(5):1403-1412). The skilled person can in particular refer to the examples of the present application to implement a thioalkylation of the ELP peptide designated ELPM80.

According to one embodiment, the alkenylated ELP implemented in the present invention comprises at least one occurrence of the amino acid sequence VPGMG in which the methionine residue is alkenylated.

According to a particular embodiment, the alkenylated ELP used to produce the composite matrix according to the invention is an ELP with a high molecular weight, in particular greater than 20 kDa.

In one embodiment, the alkenylated ELP has a structure of formula (II)

Z—[VPGXG]ₙ (II)

wherein:
Z is a peptide comprising between 1 and 20 amino acids;
X represents a glycine residue, a valine residue or an alkenylated methionine residue, in particular an alkenylated methionine residue of formula (III):

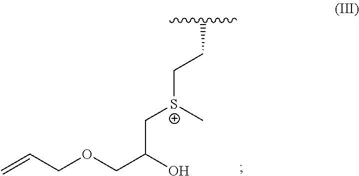

n is an integer comprised between 40 and 160 more particularly between 60 and 100, especially between 70 and 90; and
wherein the molar ratio valine/methionine alkenylated at position X is comprised between 0:1 and 10:1, more particularly between 1:1 and 5:1, especially between 2:1 and 4:1, said ratio being more particularly 3:1.

According to one embodiment, X represents a glycine residue or an alkenylated methionine residue, in particular an alkenylated methionine residue of formula (III). According to another, preferred, embodiment, X represents a valine residue or an alkenylated methionine residue, in particular an alkenylated methionine residue of formula (III).

According to one embodiment, n is an integer comprised between 60 and 100, in particular between 70 and 90, more particularly between 76 and 84, n being more particularly equal to 76, 77, 78, 79, 80, 81, 82, 83 or 84. More particularly, n is equal to 80.

According to a particular embodiment, Z is a peptide whose amino acid residue at the amino-terminal end is a methionine. According to another embodiment, the amino acids included in Z immediately upstream of the [VPGXG]ₙ unit correspond to the MW dipeptide. In particular, Z may consist of or comprise the MW dipeptide. In a particular embodiment, Z consists of the MW dipeptide.

According to one embodiment, the ELP used is a peptide of formula Z—[VPGXG]ₙ in which the molar ratio valine/methionine alkenylated at position X is comprised between 1:1 and 5:1, in particular between 2:1 and 4:1, said ratio being more particularly 3:1.

According to another embodiment, the ELP used is a peptide of formula Z—[(VPGVG)(VPGMG)(VPGVG)₂]ₓ, in particular MW[(VPGVG)(VPGMG)(VPGVG)₂]ₓ in which x is an integer comprised between 15 and 25, in particular between 19 and 21, x being more particularly equal to 20.

According to one embodiment, the ELP used is derived from the peptide MW[(VPGVG)(VPGMG)(VPGVG)₂]₂₀ (ELPM80) described in the examples, said peptide comprising at least one alkenylated methionine residue. According to another particular embodiment the ELP used is of formula MW[(VPGVG)(VPGMaG)(VPGVG)₂]₂₀ (ELPM(alkene)-80) described in the examples, in which Ma represents the alkenylated methionine residue of formula (III) above.

According to a particular embodiment, the composite matrix according to the invention comprises from 0.1 to 99.9% (w/v) ELP. According to another particular embodiment, for a composite matrix with a concentration of between 3 and 5% (w/v) and for a thiol:alkene ratio of 1:1, said matrix comprises from 1 to 4% (w/v) ELP, in particular ELPM80.

### 2. Bioactive Peptides

The composite matrix may also comprise bioactive peptides. Bioactive peptides are able to perform a biological function when the composite matrix is implanted in a tissue or organ of a subject. Such a bioactive peptide may in particular be an adhesion peptide, able to bind a cell of interest, or a calcium-binding peptide.

These bioactive peptides included in the composite matrix of the invention may comprise cysteine residues at each of their ends. The cysteine residues may be covalently linked directly to the amino acid sequence of the peptide, or via spacers, in particular a peptide or pseudopeptide spacer. In particular, the spacer can be an amino acid or an amino acid sequence (in particular a di- or tripeptide), in particular a beta amino acid, more particularly a beta-Ala amino acid.

According to a particular embodiment, the amount of bioactive peptides in the composite matrix according to the invention is comprised between 99.9 and 0.1% (w/v). According to another particular embodiment, for a composite matrix with a concentration comprised between 3 and 5% (w/v) and for a thiol:alkene ratio of 1:1, said matrix comprises from 0.1 to 3% (w/v) of the at least one bioactive peptide.

### 2.1. Adhesion Peptide

A first category of bioactive peptides that may be used in the composite matrix of the invention are adhesion peptides, able to recruit neuronal, mesenchymal and/or endothelial cells. As peptides able to recruit endothelial cells, one can mention in particular peptides derived from laminin, fibronectin or type I collagen, more particularly laminin. Hence, adhesion peptides can be selected from the fibronectin-derived peptides REDV, RGD and GRGDSP, the laminin-derived peptides IKLLI, IKVAV, PDSGR and YIGSR, and the type I collagen-derived peptide DGEA. As peptides able to recruit neuronal cells, one can mention in particular the laminin-derived peptides YIGSR, RNIAEIIKDI and IKVAV.

In a preferred embodiment, the composite matrix comprises at least one adhesion peptide selected from IKVAV and YIGSR.

According to a particular embodiment, the adhesion peptide is an IKVAV peptide. The IKVAV peptide may in particular be an IKVAV peptide of formula Cys-{spacer}-Ile-Lys-Val-Ala-Val-{spacer}-Cys, in particular the peptide of formula Cys-{Beta-Ala}-Ile-Lys-Val-Ala-Val-{Beta-Ala}-Cys.

According to another particular embodiment, the adhesion peptide is a YIGSR peptide. The YIGSR peptide may in particular be a YIGSR peptide of the formula Cys-{spacer}-Tyr-Ile-Gly-Ser-Arg-{spacer}-Cys, in particular the peptide of the formula Cys-{Beta-Ala}-Tyr-Ile-Gly-Ser-Arg-{Beta-Ala}-Cys.

According to an even more preferred embodiment, the composite matrix comprises the IKVAV peptide and the YIGSR peptide.

According to a preferred alternative embodiment, in order to optimize and control crosslinking of the composite matrix, the latter comprises a single adhesion peptide comprising at least two adhesion peptides, in particular a biomimetic peptide comprising the adhesion peptides IKVAV and YIGSR.

The peptides making up the single adhesion peptide can be included in any order. According to an alternative embodiment, the single adhesion peptide comprises, from the amino terminal end to the carboxy terminal end, the peptides YIGSR and IKVAV. In a preferred alternative embodiment, the single adhesion peptide comprises the peptides IKVAV and YIGSR from the amino terminal end to the carboxy terminal end. The single adhesion peptide may comprise cysteine residues at each of its ends. The cysteine residues may be covalently linked directly to the amino acid sequence of the single adhesion peptide, or via spacers, in particular a peptide or pseudopeptide spacer. The spacer can in particular be an amino acid or an amino acid sequence (in particular a di- or tripeptide), in particular a beta amino acid, more particularly a beta-Ala amino acid. Moreover, the peptides making up the single adhesion peptide can be covalently linked to each other directly or via spacers. The spacer between the peptides making up the single adhesion peptide can in particular be a peptide or pseudopeptide spacer, in particular a glycine amino acid, a diglycine or a triglycine.

Furthermore, the single adhesion peptide comprising at least two adhesion peptides may also advantageously include one or more target sequences of one or more metalloproteases, in particular metalloproteases 2 and 9. By way of illustration, one can cite the peptides:
PVGLIG;
peptides QPQGLAK, GPLGLSLGK and GPLGMHGK described in Jha et al., Biomaterials, May 2016; 89; p. 136-47; and
peptides GPQGIAGQ, GPQGIWGQ and VPMSMRGG described in Lin et al., Acta Biomaterialia, 10(12), December 2014, p. 5106-5115.

Such a metalloprotease target peptide can be introduced to enable cleavage between adhesion peptides, which remain bound to the matrix of the invention while being available for cell adhesion. Moreover, a metalloprotease target peptide may be useful for triggering matrix degradation, thereby facilitating cell colonization. A metalloprotease target peptide can be included in the single adhesion peptide at any position, in particular at its amino terminal end, between two adhesion peptides included in the single adhesion peptide, or at its carboxy terminal end. Preferably, the metalloprotease target peptide(s) are positioned between two adhesion peptides in the single adhesion peptide.

According to a particular embodiment, the single adhesion peptide includes at least one occurrence, in particular a single occurrence, of the target sequence of metalloproteases 2 and 9 PVGLIG.

According to a particular embodiment, the composite matrix comprises a single adhesion peptide comprising the peptides IKVAV, PVGLIG and YIGSR. According to a particular embodiment, said single adhesion peptide is of formula:

IKVAV(spacer)PVGLIG(spacer)YIGSR;
YIGSR(spacer)PVGLIG(spacer)IKVAV;
PVGLIG(spacer)YIGSR(spacer)IKVAV;
PVGLIG(spacer)IKVAV(spacer)YIGSR;
IKVAV(spacer)YIGSR(spacer)PVGLIG;
or
YIGSR(spacer)IKVAV(spacer)PVGLIG.

Preferably, the single adhesion peptide is of the formula IKVAV(spacer)PVGLIG(spacer)YIGSR.

The spacers designated “(spacer)” in the unique sequences listed above may be of the same or different sequence, in particular identical. In particular, a spacer may be selected from a glycine residue, a diglycine dipeptide or a triglycine tripeptide.

According to one alternative embodiment, said single adhesion peptide is of formula:

IKVAV-GGG-PVGLIG-GGG-YIGSR;
YIGSR-GGG-PVGLIG-GGG-IKVAV;
PVGLIG-GGG-YIGSR-GGG-IKVAV;
PVGLIG-GGG-IKVAV-GGG-YIGSR;
IKVAV-GGG-YIGSR-GGG-PVGLIG;
or
YIGSR-GGG-IKVAV-GGG-PVGLI.

Preferably, said single adhesion peptide is of the formula IKVAV-GGG-PVGLIG-GGG-YIGSR.

Prior to cross-linking and formation of the composite matrix, the peptide enabling introduction of the single peptide into said composite matrix may be of formula:

CBA-IKVAV(spacer)PVGLIG(spacer)YIGSR-βAC;
CBA-YIGSR(spacer)PVGLIG(spacer)IKVAV-βAC;
CBA-PVGLIG(spacer)YIGSR(spacer)IKVAV-βAC;
CBA-PVGLIG(spacer)IKVAV(spacer)YIGSR-βAC;
CBA-IKVAV(spacer)YIGSR(spacer)PVGLIG-βAC;
or
CBA-YIGSR(spacer)IKVAV(spacer)PVGLIG-βAC.

Preferably, prior to cross-linking and formation of the composite matrix, the peptide enabling introduction of the single peptide into said hydrogel may be of formula

CβA-IKVAV(spacer)PVGLIG(spacer)YIGSR-βAC.

In a preferred embodiment, prior to crosslinking and formation of the composite matrix, the peptide enabling introduction of the single peptide into said composite matrix is of the formula:

CBA-IKVAV-GGG-PVGLIG-GGG-YIGSR-βAC;
CBA-YIGSR-GGG-PVGLIG-GGG-IKVAV-βAC;
CBA-PVGLIG-GGG-YIGSR-GGG-IKVAV-βAC;
CBA-PVGLIG-GGG-IKVAV-GGG-YIGSR-βAC;
CBA-IKVAV-GGG-YIGSR-GGG-PVGLIG-βAC;
or
CBA-YIGSR-GGG-IKVAV-GGG-PVGLIG-βAC.

Particularly preferably, the peptide prior to cross-linking and formation of the composite matrix, the peptide enabling introduction of the single peptide into said composite matrix is of the formula

CBA-IKVAV-GGG-PVGLIG-GGG-YIGSR-βAC.

In a particular embodiment, the amount of peptide used to introduce the single peptide into the composite matrix according to the invention is between 99.9 and 0.1% (w/v). Depending on the composition of the single peptide, and the presence of other bioactive peptides, and for a given composite matrix concentration, the person skilled in the art will of course be able to adjust the quantity of the single peptide.

### 2.2. Peptides Able to Induce Calcium Phosphate Nucleation

Peptides able to induce calcium phosphate nucleation constitute a second category of bioactive peptides that may be introduced into the composite matrix according to the invention. The experiments provided below show that in a composite matrix according to the invention, distribution homogeneity and retention of calcium phosphate are improved in the presence of such a peptide able to induce calcium phosphate nucleation. It is in particular shown that, unexpectedly, in the presence of the SNA15 peptide, around 10 times more hydroxyapatite particles are found in the matrices, which can have a beneficial effect on the osteoconductive and/or osteoinductive properties of the composite matrices.

For the purposes of composite matrix formation, they may be included in a single peptide also comprising at least one adhesion peptide, or constitute an independent peptide. In a preferred embodiment, the peptide able to induce calcium phosphate nucleation is a peptide that is not bound to an adhesion peptide, prior to formation of the composite matrix.

By way of illustration, one can mention, among peptides able to induce calcium phosphate nucleation:
peptides derived from statherin, in particular DDDEEKFLRRIGRFG (SNA15), and SNA15 analogs, more particularly peptides DSSEEKFLRRIGRFG (SNS15) and EFLRRIGRFG (SN11) described in Raj et al. JBC, volume 267(9), Mar. 25 1992, Pages 5968-5976;
peptide DHTKE (Sun et al. Journal of Agricultural and Food Chemistry 2017 65 (44), 9782-9789);
peptide SSSEEIVPN (Meisel et al. Biol Chem Hoppe Seyler. December 1988; 369(12):1275-9);
peptide DEGEQPRPFPFP (Lv et al. Food Chemistry, Volume 141(3), 2013, Pages 1645-1650);
peptide WEWLHYW (Charoenphun et al. Eur Food Res Technol 236, 57-63 (2013));
peptide DGDDGEAGKIG (Chen et al. Journal of Functional Foods, Volume 6, 2014, Pages 575-584);
peptide GPAGPHGPPG (Guo et al. Food Chemistry, Volume 173, 2015, Pages 536-542);
peptide VLSGGTTMYASLYAG (Jung et al. Eur Food Res Technol 224, 763-767 (2007));
peptide TCH (Huang et al. Eur Food Res Technol 232, 281-287 (2011));
peptide VLGYIQIR. (Hou et al. Food Chemistry, Volume 243, 2018, Pages 389-395); and
peptide DNLPNPEDNKNYQ (Choi et al. Food Sci Biotechnol 21, 1663-1667 (2012)).

According to a preferred embodiment, the peptide able to induce calcium phosphate nucleation is a peptide derived from statherin, more particularly an SNA15 analog, even more particularly a peptide selected from SNA15, SNS15 and SN11. Preferably, the peptide able to induce calcium phosphate nucleation is the SNA15 peptide.

The peptide able to induce calcium phosphate nucleation may comprise cysteine residues at each of its ends. Cysteine residues can be covalently linked directly to the amino acid sequence of the peptide, or via spacers, in particular a peptide or pseudopeptide spacer. In particular, the spacer can be an amino acid or an amino acid sequence (in particular a di- or tripeptide), in particular a beta amino acid, more particularly a beta-Ala amino acid. According to a preferred embodiment, prior to crosslinking and hydrogel formation, the peptide enabling the introduction of the peptide able to induce calcium phosphate nucleation into the composite matrix of the invention may be of the formula CβA-DDDEEKFLRRIGRFG-βAC.

According to a particular embodiment, the amount of peptide able to induce calcium phosphate nucleation in the composite matrix according to the invention is comprised between 99.9 and 0.1% (w/v). As noted above, according to a particular embodiment, for a composite matrix with a concentration of between 3 and 5% (w/v) and for a thiol:alkene ratio of 1:1, said matrix may in particular comprise from 0.1 to 3% (w/v) of at least one bioactive peptide.

### 3. Calcium Phosphate

The composite matrix according to the invention also includes a mineral phase comprising calcium phosphate. Illustrative examples include calcium phosphate in the form of apatite, tri-calcium phosphate, bi-calcium phosphate and mixtures thereof. These calcium phosphate sources can be used in any available crystalline form. Moreover, derivatives of these calcium phosphate sources, in particular carbonaceous derivatives, may also be employed. According to a particular embodiment, said calcium phosphate is in the form of apatite, more particularly hydroxyapatite. Advantageously, such a calcium phosphate source allows to enhance the osteogenic properties of the composite matrix.

According to a particular embodiment, the composite matrix according to the invention comprises between 0.1 and 50% (w/v) calcium phosphate. According to a particular embodiment, the composite matrix according to the invention comprises between 0.1 and 50% (w/v) hydroxyapatite. More particularly, the composite matrix may comprise between 0.2 and 20% (w/v) hydroxyapatite, more particularly between 0.5 and 4% (w/v), in particular between 2 and 3% (w/v). According to a particular embodiment, the composite matrix comprises 2.5% (w/v) hydroxyapatite.

According to another particular embodiment, the composite matrix according to the invention comprises between 1 and 3% (w/v), more particularly 2% (w/v) hydroxyapatite.

### 4. Illustrative Embodiments of the Composite Matrix According to the Invention

In particular, the composite matrix according to the invention may comprise an elastin-like peptide, at least one bioactive peptide, and calcium phosphate.

According to one embodiment, the concentration of composite matrix is comprised between 1 and 10% by density (w/v), in particular between 3 and 5% (w/v).

According to another embodiment, the thiol:alkene molar ratio is comprised between 3:1 and 1:3, more particularly between 2:1 and 1:2, this ratio being more particularly equal to 1:1.

According to a particular embodiment, the composite matrix comprises:
(i) an elastin-like peptide;
(ii) at least one adhesion peptide;
(iii) at least one peptide able to induce calcium phosphate nucleation; and
(iv) calcium phosphate.

Components (i) to (iv) may be selected from the components described in parts 1 to 3 above.

More particularly, the composite matrix according to the invention may comprise:
(i) a peptide derived from the elastin-like peptide ELPM80;
(ii) a peptide IKVAV and a peptide YIGSR;
(iii) the peptide SNA15 or a peptide analogous to SNA15; and
(iv) hydroxyapatite.

Preferably, the composite matrix according to the invention comprises:
(i) a peptide ELPM80;
(ii) a peptide IKVAV(spacer)PVGLIG(spacer)YIGSR;
(iii) the peptide SNA15; and
(iv) hydroxyapatite.

More preferably, the composite matrix according to the invention comprises:
(i) a peptide ELPM80;
(ii) a peptide IKVAV-GGG-PVGLIG-GGG-YIGSR;
(iii) the peptide SNA15; et
(iv) hydroxyapatite.

### 5. Process for Preparing the Composite Matrix

The composite matrix of the invention can be manufactured by mixing its various components, and any other optional elements. The components of the composite matrix and the quantity of these components are selected in order to prepare a composite matrix with physical and support properties adapted to the user's requirements.

The person skilled in the art is familiar with the crosslinking techniques employed in the state of the art to manufacture a composite matrix by crosslinking. The composite matrix can thus be made by crosslinking under the action of a stimulus such as a change in temperature or pH, or by means of a crosslinking agent, in particular a photosensitive crosslinking agent (or photoinitiator). As an illustrative example, one can mention induction of photopolymerization by means of a photoinitiator such as the compound Irgacure 2959, in particular used at a density of 0.5% (w/v) in the mixture, and activated by UV-visible light (λ=305-405 nm, in particular 305 nm) between 5 and 12 minutes, more particularly between 6 and 10 minutes, in particular for about 8 minutes. In another alternative, the photoinitiator can be selected from lithium phenyl-2,4,6-trimethylbenzoylphosphinate (LAP) and riboflavin. The concentration of LAP can vary from 0.005% to 0.5% (w/v) in the mixture, and its photoinitiation can be triggered at a wavelength comprised between 365 and 475 nm for 5 to 12 minutes, more particularly between 6 and 10 minutes, especially around 8 minutes.

Advantageously, the skilled person may also refer to the preparation process described in the examples of the present application, in which the hydrogel is produced by forming a cryomatrix. In one alternative embodiment, a prematrix solution comprising the various matrix components is frozen to form water crystals. As the crosslinking reagents (i.e. the components carrying thiol and alkenyl moieties) surround said water crystals, and when the solution is subjected to UV radiation, the alkene and thiol moieties of the various components of the mixture react at the interface of said crystals. When the solution thaws, the space occupied by the water crystals becomes a pore in the matrix. This step may be followed by a freeze-drying step. For example, after crosslinking, matrices can be rehydrated, in particular for 24 hours, and freeze-dried under vacuum after deep freezing, thus sublimating the ice without melting it, and generating new pores. The combination of these two techniques has the advantage of enabling the formation of a wide distribution of pore sizes, allowing colonization of different cell types useful for inducing angiogenesis, innervation and osteogenesis.

Thus, according to a particular embodiment of the process according to the invention, the freezing step is carried out at a temperature of between 0 and −80° C., more particularly at a temperature of about −20° C., for at least 2 h, in particular for about 24 h. The prematrix can then be subjected to UV radiation suitable for inducing photopolymerization, in particular for about 5 to 12 minutes, more particularly for about 6 to 10 minutes, in particular for about 8 minutes. After crosslinking, and according to a particular embodiment, a rehydration step can be carried out, in particular for at least 2 h, in particular for about 24 h. This rehydration step may be followed by a deep-freezing step at around −80° C. for at least 1 h, in particular for around 24 h, then by a freeze-drying step lasting around 24 to 48 h.

As mentioned above, these steps enable the formation of a composite matrix comprising pores with a wide, controlled pore size distribution. The composite matrix according to the invention can in particular have macropores with an average size greater than 100 μm

### 6. Use of the Composite Matrix According to the Invention

Advantageously, the properties of the composite matrix can be very finely defined. Furthermore, said composite matrix according to the invention is biodegradable and has a porous structure suitable for a colonization by various cell types useful for the induction of angiogenesis, innervation and osteogenesis. The inventors could also show that the composite matrix according to the invention is not cytotoxic. It thus brings together all the advantageous properties useful for the development of a tool suitable for tissue regeneration.

The composite matrix according to the invention is therefore able to support efficiently in vitro culture of different cell types. Accordingly, in a particular embodiment, the invention relates to a new three-dimensional support able to host in vitro various cells of interest for bone regeneration, in particular neuronal, bone or endothelial cells. The invention thus provides the person skilled in the art with a particularly advantageous 3D cell culture system enabling cells to develop in a favorable environment, but also to study interactions of different cell types with each other. This parameter is important for studying regeneration phenomena, which may require complex dialogues between different cell types. The carrier of the invention can in particular be used to accommodate osteoforming and endothelial cells, and to study the angiogenic, osteogenic and innervation effect in an in vitro cell culture method, comprising culturing cells in a carrier as defined above. The use of the support according to the invention can also include the addition of an agent to the culture, such as a growth factor or any other agent having a biological effect or likely to have a biological effect (candidate agent) to determine its effect on one or more parameters and cellular responses such as cell growth, induction of quiescence, cell death, secretion of protein or other molecules or ions (in particular calcium or potassium ions), or else the expression of some genes.

According to another aspect, the composite matrix of the invention is used in a treatment method, in particular as an implant. In particular, the composite matrix is used in a regenerative medicine treatment method. It can in particular be used to stimulate the innervation of a tissue, especially bone tissue, and is especially suitable for use in bone engineering. Advantageously, the composite matrix according to the invention promotes innervation, particularly in a regeneration context. More particularly, the composite matrix according to the invention can advantageously be used to recruit and stimulate the sensory nervous system, more particularly to promote bone regeneration. The composite matrix according to the invention can also be used to optimize or restore vascularization and innervation of a tissue.

According to another embodiment, the composite matrix according to the invention is used to repair complex lesions that exhibit vascular and nerve damage.

Furthermore, the composite matrix according to the invention can also be used to regenerate the interface with the peripheral nervous system, and to control bone development and repair.

According to another embodiment, the composite matrix can be used as a cell therapy vehicle. Thus, a composite matrix as defined above can be previously colonized with cells of therapeutic interest, for example by means of stem cells, in particular induced stem cells in a lineage of interest, hematopoietic stem cells, mesenchymal stromal stem cells derived from bone marrow or adipose tissue, neural stem cells, or a mixture of cells of different lineages. Such a composite matrix can be used in a cell or tissue regeneration treatment method.

The composite matrix according to the invention can also be used to modify implant systems, but also to improve their biocompatibility and integration.

### LEGEND OF THE FIGURES

FIG. 1: Fluorescence scattering and pore size quantification of hydrated matrices. A) ELPM80+pore size quantification of hydrated hydrogels YIGSR. N=2-5 hydrogels (502<pores<2100) Kruskal-Wallis; Dunn's post-hoc; p<0.001. C) Pore size classification of hydrogels ELPM80+YIGSR into micropores (required for angiogenesis and innervation) and macropores (required for bone formation).

FIG. 2: Quantification of vessels and nerve structures formed within the hydrogel structure of the composition IKVAV+YIGSR and its scramble version VKAIV+GYSRI. Each implantation is represented by a dot and the mean is represented by a bar (n=5-6). Note that within the same group, a high variability is observed, confirming heterogeneous cell colonization within the hydrogel structure. No statistical differences were detected.

FIG. 3: Matrix production steps: (1) preparation of pre-matrix solutions, frozen at −20° C. for 24 hours before (2) cross-linking under UV light. (3) The cryogel is hydrated for 24 hours before (4) freeze-drying.

FIG. 4: Quantification of structure and pore size of matrices dehydrated to 4% (w/v) with 0% or 5% (w/v) HA. Pore size was quantified using ImageJ. Data is shown as +SD, 10<n<32, with statistical differences indicated *** p<0.001 (ANOVA with post-hoc Bonferroni test).

FIG. 5: Structure and pore size of dehydrated matrices produced at 8 min crosslinking time with different final mass concentrations (% w/v) and different HA concentrations (% w/v). Pore size quantification. Data is represented as means±SD, 7<n<26, no statistical differences were observed (ANOVA with Bonferroni test as post-hoc test or Mann-Whitney test).

FIG. 6: Internal porosity of matrices hydrated at 3% (w/v) and 4% (w/v) with different HA concentrations. (B) Pore size was quantified using ImageJ. Data is represented as +SD, 21<n<65, with statistical differences indicated by * p<0.05 and *** p<0.001 (ANOVA with Bonferroni test, post-hoc). Bars: 100 μm.

FIG. 7: Mineralization potential of 3% w/v matrices without HA or containing 1%, 2% and 2.5% w/v HA implanted subcutaneously in mice for 2 and 4 weeks. Quantification of microCT acquisitions using MicroView software on day 0 (day of implantation), and at 2 and 4 weeks. Note that the volume of mineralized tissue increases with time, and that matrices containing 2% and 2.5% w/v HA produced a higher volume of mineralized tissue than matrices without HA and containing 1% w/v HA. Data is represented as mean±SD, n=4-5 mice per group, with statistical differences indicated by * p<0.05 and ** p<0.01 (ANOVA with Bonferroni test, post-hoc).

FIG. 8: Scanning electron microscopy coupled with EDX analysis on ELPs matrices+IKVAV/YIGSR peptides containing or not the peptide SNA 15. ELPs Matrices+IKVAV/YIGSR peptides containing 0, 1, 2 and 2.5% w/v HA containing or not the peptide SNA 15 were lyophilized and cut in half. The inside of the cut matrices was analyzed. The signal in the form of white dots corresponds to the calcium particles making up hydroxyapatite. The bars shown in the figure correspond to 2 mm.

FIG. 9: Longitudinal micro-CT monitoring of matrix mineralization. Representative micro-CT images of subcutaneous implantation of matrices without HA or containing 1%, 2% and 2.5% (w/v) HA after 0, 15 and 30 days of implantation. The ratio mineral volume/total volume (MV/TV) was measured from three-dimensional micro-CT images reconstructed using Microview® software. The ANOVA statistical test followed by Bonferroni's post hoc test with * p<0.05 and ** p<0.01 was performed.

FIG. 10: Micro-CT monitoring of mineralization of ELPs matrix containing 2% (w/v) HA implanted subcutaneously using MicroView® software.

FIG. 11: Histological analysis of ectopically implanted matrices stained with Masson's Trichrome. Matrices containing 0%, 1%, 2% and 2.5% HA were implanted subcutaneously and stained with Masson's Trichrome. Quantitative analysis of osteoid tissues formed (15<n<23 per sample) with a Kruskal-Wallis multiple comparison test with p ** <0.01 and p *** <0.001.

FIG. 12: Immunostaining of vascular networks and nerve structures in composite matrices containing 2% HA after 0, 3, 7, 15 and 30 days of implantation in ectopic sites. (A) Quantification of surface area occupied by blood vessels relative to the matrix surface area×1000. (B) Quantification of surface area occupied by nerve structures relative to the matrix surface area×1000. Quantitative analysis with a Kruskal-Wallis multiple comparison test

FIG. 13: Micro-CT analysis of mineralization induced by composite matrices in a femoral condyle bone lesion model in rat. (A) Micro-CT images representative of bone lesions after 7, 15, 30 and 60 days of implantation and according to groups: lesion left empty, filled with ELPs matrices containing 2% HA and the positive control Collapat®. (B) The ratio mineral volume/total volume (MV/TV) was measured from three-dimensional micro-CT images reconstructed using Microview® software for the three groups (empty, ELPs, Collapat®) and for all implantation times (D7, D15, D30 and D60). Quantitative analysis of mineralization with a Kruskal-Wallis multiple comparison test with p * <0.1 and ns=not significant.

FIG. 14: Histological section of a lesion in the femoral condyle filled with ELP matrix/peptides containing 2% HA. Staining with Masson's Trichrome of a bone lesion filled with the composite matrix after 7 days of implantation. The area corresponding to the lesion is delimited by a black dotted circle.

FIG. 15: Matrix mineralization in the mandibular lesion model. 3D micro-CT images showing the reconstruction of the mandibular defect in the presence of the composite matrix.

### EXAMPLES

### Example 1: Preparation of a Hydrogel with Improved Properties

The inventors' team recently evaluated a biomaterial taking into account the importance of vascularization and innervation and during bone regeneration (Paiva dos Santos et al., Acta Biomater. 2019, 99, 154). The aim of this work was to develop an acellular, growth factor-free hydrogel that promotes angiogenesis and innervation. Hydrogels comprising ELPM40, polyethylene glycol (PEG), and different concentrations of the adhesion peptide IKVAV (25% w/w and 50% w/w) were produced, characterized and evaluated. In vitro tests could show that the 50% IKVAV composition had greater potential to support osteogenesis, angiogenesis and innervation. In vivo, this composition did not induce any inflammatory response, and allowed to induce a higher vascular density and formation of nerve endings in surrounding tissues, after subcutaneous implantation in mice. However, this previous study has some limitations. Firstly, the porosity of the hydrogel was not sufficient to allow a homogeneous cell colonization in the implant. Furthermore, the hydrogel does not appear to degrade after implantation, probably because PEG is not fully biodegradable in the long term.

The inventors therefore sought to modify the composition and structure of the hydrogel in order to improve the homogeneity of cell colonization and the biodegradability of the material.

### Development of a Method for Inducing Improved Porosity

Firstly, the inventors sought the best method for inducing porosity in hydrogel matrices and enabling their colonization by cells. These structures were analyzed by scanning electron microscopy (SEM).

State-of-the-art hydrogels composed of ELPM40 and PEG were produced, and their pore sizes were characterized.

The first method used for inducing pores, called “CryoUV” below, involves the formation of a cryomatrix. A prematrix solution was frozen to form water crystals. As the crosslinking reagents surround said water crystals, and when the solution is subjected to UV radiation, the alkene and thiol moieties of the various components of the mixture react at the interface of said crystals. When the solution thaws, the space occupied by the water crystals becomes a pore in the matrix.

The other method used is based on the production of matrices using the CryoUV method, followed by a freeze-drying step (referred to below as “CryoUV+Lyoph”). After cross-linking, the matrices were rehydrated for 24 h, and freeze-dried under vacuum after deep freezing for 24 h, thus sublimating the ice without melting it, and thus generating new pores. The combination of these two techniques has the advantage of enabling the formation of a wide distribution of pore sizes, allowing the colonization of different cell types useful for the induction of angiogenesis, innervation and osteogenesis.

The inventors assessed the external structure of the matrices using environmental SEM. CryoUV hydrogels were produced and, prior to SEM analysis, their water content was evaporated to enable SEM visualization. For the CryoUV+Lyoph method, freeze-drying enabled the three-dimensional structure of the hydrogel to be preserved. The results show that both methods enable the production of hydrogels with a porous structure.

The inventors then explored the possibility of modifying the hydrogel composition to improve the above-mentioned colonization and biodegradability properties.

### Production of Peptide ELPM(Alkene)-80

Plasmid ELPM40-pUC19 encoding ELPM40 MW[(VPGVG)(VPGMG)(VPGVG)₂]₁₅ described previously (Petitdemange et al., Biomacromolecules. 2017 Feb. 13; 18(2):544-550) was digested by the restriction enzyme BsmF1 to linearize it, then 5′ dephosphorylated by the phosphatase Antarctic. The nucleotide sequence encoding the amino acid sequence [(VPGVG)(VPGMG)(VPGVG)₂]₅ was extracted from a plasmid pUC19 encoding ELPM20, also described above, by digestion with the restriction enzymes BsmF1 and BtgZI. This sequence was used as an insert introduced by ligation into the plasmid ELP M40-pUC19 linearized, thus obtaining plasmids pUC19-ELPM60 and pUC19-ELPM80 encoding ELPM60 and ELPM80 respectively. The sequence encoding ELPM80 was then transferred, after digestion of plasmid pUC19-ELPM80 with the restriction enzymes NdeI and BamHI, into the expression vector pET44a, enabling an IPTG-inducible expression of ELPM80. The article of Petitdemange et al. (Biomacromolecules. 2017 Feb. 13; 18(2):544-550) describes the construction of the expression vector of peptide MX[VPGVGVPGMG(VPGVG)₂]₁₀ (ELPM40), its expression in E. coli, its isolation from bacterial lysates, its purification and its characterization. These conditions were adapted for the production and purification of ELPM80. Correct structure and molecular mass were confirmed by 1H NMR and MALDI mass spectrometry.

An alkene moiety was then added to the peptide ELPM80 by chemoselective thioalkylation of the methionine side chains of the peptide ELPM80, again according to the conditions described in Petitdemange 2017, using allyl glycidyl ether to produce the peptide ELPM(alkene)-80, designated ELPMa-80 later. The structure of the resulting alkenylated peptide was confirmed by 1H NMR.

### Production and Characterization of a Composite Matrix Comprising ELPM80

In order to improve the hydrogel composition, the inventors replaced ELPM40 included in prior art compositions with the elastin-like peptide ELPM80. Incidentally, the adhesion peptide YIGSR was also introduced into the hydrogel composition. Its scramble version GYSRI was used as a control. The hydrogel compositions produced are shown in Table 1.

TABLE 1
Hydrogel compositions comprising ELPM80
and the adhesion peptide YIGSR

| Groups | Component | Concentration | |
|---|---|---|---|
| ELPM80 + | ELPM80 | 1.64 | mM |
| 100% YIGSR | YIGSR or scramble | 16.68 | mM |
| ELMP80 + | ELPM80 | 1.64 | mM |
| 50% IKVAV + | IKVAV or scramble | 8.62 | mM |
| 50% YIGSR | YIGSR or scramble | 8.62 | mM |

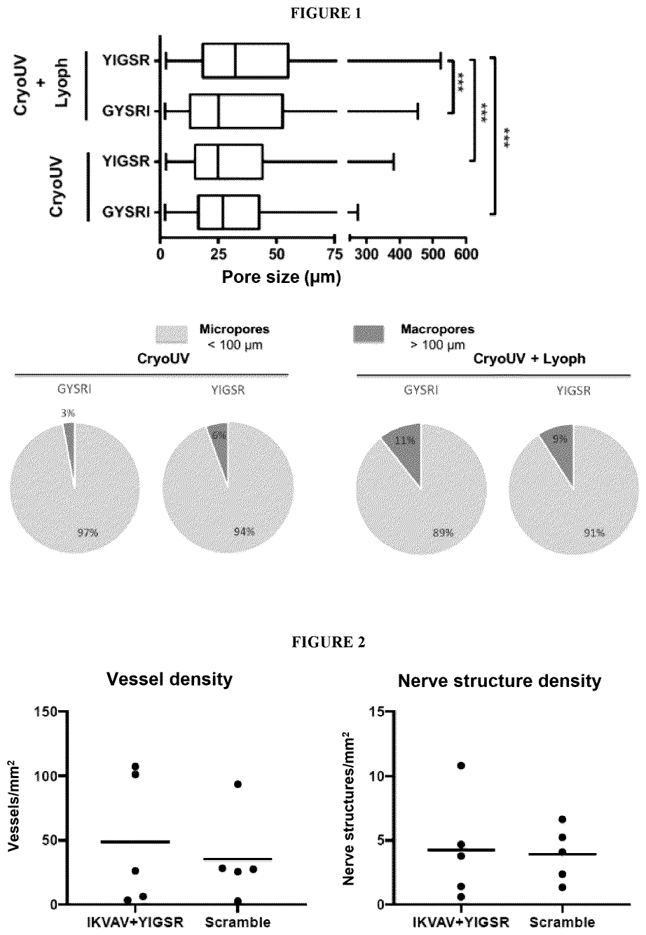

Diffusion of FITC-dextran 500 kDa into the matrix structure and assessment of fluorescence in the core material by confocal microscopy were used to quantify pore size and porosity using ImageJ. The lowest measured pore size for both the CryoUV and CryoUV+Lyoph methods is 2.16 μm, and the highest is 382.5 μm with the CryoUV method, and 523.5 μm with the CryoUV=Lyoph method, indicating that the latter produces larger pores (FIG. 1).

In order to satisfy angiogenesis, innervation and bone formation, the range of pores needs to be very wide. Data from the literature suggest that the optimal size is 0.005 to 0.3 μm for innervation, 5 to 15 μm for fibroblast growth, 50 to 400 μm for rapid vascularization, and 100 to 400 μm for bone regeneration. Taking into account pore size to induce bone formation, we assessed the pore frequency according to size, representing them into micropores (smaller than 100 μm) and macropores (larger than 100 μm). In general, macropores were more frequent when samples had been freeze-dried compared to the CryoUV method (FIG. 1), which satisfies angiogenesis, innervation and bone regeneration.

Based on the previous section, the CryoUV+Lyoph method was standardized to ensure (i) high porosity taking into account criteria of (ii) large pore sizes to satisfy the prerequisite of cell colonization by different cells enabling angiogenesis, innervation and osteogenesis; iii) compositional modifications were made to produce an all-natural polymer material in order to facilitate matrix degradation, meaning that production of a PEG-free matrix was possible, while maintaining the alkene/thiol equimolarity; iv) ELPM40 was replaced by ELPM80. The inventors implanted the IKVAV+YIGSR composition and also its scramble version subcutaneously in mice to assess angiogenesis and innervation potential in vivo. After 4 weeks of implantation, samples were harvested and analyzed histologically by hematoxylin-eosin (HE) staining to assess tissue architecture, cell colonization and persistent inflammation. For the angiogenesis potential, a CD31 immunohistochemistry (IHC) was performed and, for innervation potential, a β3 tubulin IHC was used. For both compositions, no persistent signal of inflammation was observed, and nerves and vessels were seen inside the hydrogels. However, a heterogeneous cell colonization did occur, indicating the need to optimize and standardize material production. A quantification of vessel and nerve densities also confirmed these observations, showing a great variability within the same group (FIG. 2).

Based on the data outlined above, modifications seemed necessary to increase the functionality of the hydrogel in vivo. Given the limited reproducibility of hydrogels and the resulting cell colonization, the inventors decided to optimize the matrix production protocol and modify the composition in order to combine the osteogenic potential in a new generation of materials. As mentioned above, using hydrogels based on ELP and adhesion peptides, the inventors have already shown that hydrogels were able to induce the angiogenesis and innervation surrounding the implantation site.

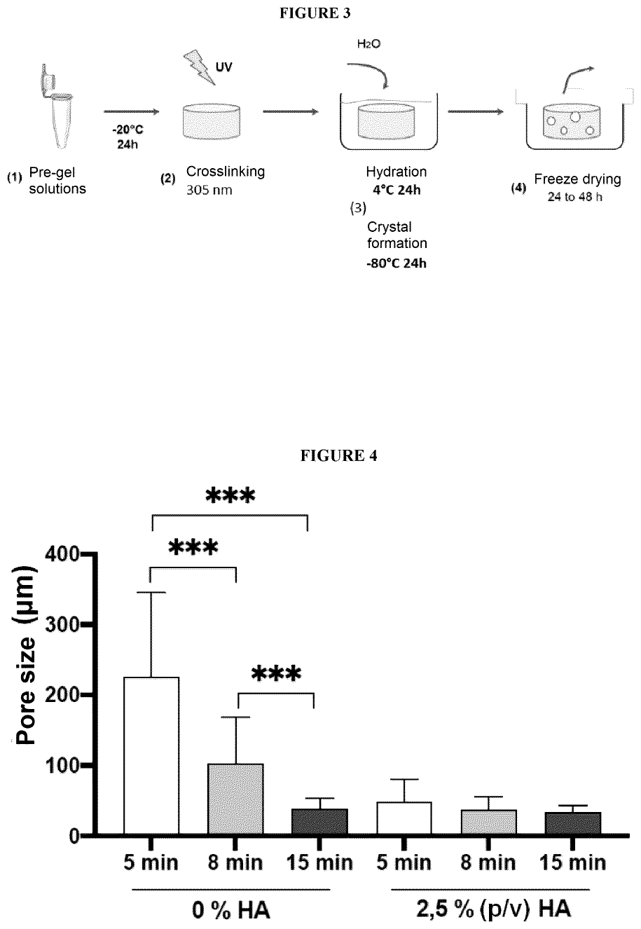

In order to increase biological functionality, the inventors considered other biomimetic peptides to enhance neural and vascular cell recruitment, matrix degradation, cell colonization and mineral content retention: (i) the basic adhesion peptides CβA-IKVAV-βAC and CβA-YIGSR-βAC were replaced by the sequence COA-IKVAV-GGG-PVGLIG-GGG-YIGSR-βAC, where the same cross-linking chemistry strategy was retained: the thiol group is present on the flanking cysteines, enabling a cross-linking with the alkenes grafted onto ELPM80. The inventors also used a control peptide where the scramble versions VKAIV and GYSRI replaced their original sequences IKVAV and YIGSR, respectively. The sequence PVGLIG was retained in this control peptide. Three glycines were used as spacers between the functional units and (ii) PVGLIG, a target degradation site for matrix metalloproteinases 2 and 9, was included between the two adhesion sequences. Thus, once cleaved, each adhesion sequence will still be attached to the matrix and available for cell attachment. In addition, this sequence will trigger matrix degradation, and thus aid cell colonization. (ii) The peptide SNA15, CβA-DDDEEKFLRRIGRFG-βAC, a calcium phosphate nucleation unit derived from statherin, was used to retain and ensure a homogeneous distribution of hydroapatite particles. Consequently, (iii) the hydroxyapatite (HA), corresponding to the synthetic bone mineral content, is included in the material composition to provide biochemical signals for osteoconduction properties. The inventors' group has already synthesized and characterized them, having 50-100 nm rod-shaped HA crystals that form aggregates with an average diameter of 3.26±0.62 μm. Using the CryoUV+Lyoph method, the inventors standardized the incubation time in the matrix production protocol (FIG. 3).

Given the importance of porosity and pore size, the next step in the characterization process was to optimize the matrices according to several factors: crosslinking time, final mass concentration and HA concentration. Photopolymerization was induced using the photoinitiator Irgacure 2959, in particular used at a density of 0.5% (w/v) in the mixture, and activated by UV-visible light at 305 nm. Firstly, the crosslinking time was evaluated, because it has a direct effect on the creation of matrixes. 4% (w/v) matrices composed of ELPM80, biomimetic peptide containing the sequences IKVAV, PVGLIG and YIGSR, SNA15 and containing or not HA (Table 4) were produced, crosslinked for 5 min, 8 min and 15 min. Using a scanning electron microscopy, images from the matrices were obtained and the pore size could be quantified (FIG. 4).

TABLE 4
Matrix compositions with concentrations of all components.

| | 4% (p/v) | 3% (p/v) | 2% (p/v) |
|---|---|---|---|
| ELPM80 | 0.67 mM | 0.51 mM | 0.34 mM |
| Biomimetic | 3.54 mM | 2.65 mM | 1.77 mM |
| peptides * | | | |
| SNA15 | 3.54 mM | 2.65 mM | 1.77 mM |
| HA | 2.5-10% (p/v) | 2.5-10% (p/v) | 2.5-10% (p/v) |

* Biomimetic peptides consist of a single peptide containing the units IKVAV, PVGLIG and YIGSR. The scramble version consists of VKAIV and GYSRI without affecting the sequence PVGLIG.

The obtained results suggest that the shorter the cross-linking time (5 min), the larger the pores. Indeed, with a cross-linking time of 15 minutes, few pores were observed. Without wishing to be bound by any theory, the inventors hypothesized that the longer the crosslinking time, the more likely cryomatrixes are to start thawing. This would result in stiffer matrices, due to a higher crosslinking rate, and smaller pores due to thawing. Indeed, this may explain why, with a crosslinking time of 15 minutes, very few pores could be observed. In general, pore size differs as a function of crosslinking time in matrices without HA, but there is no difference in pore size with 5% (w/v) HA matrices. This suggests that HA interferes with pore formation, irrespective of crosslinking time. Given the pore sizes in matrices with and without HA, and the fact that the matrices were dehydrated, the inventors decided to standardize the cross-linking time to 8 minutes for the subsequent experiments.

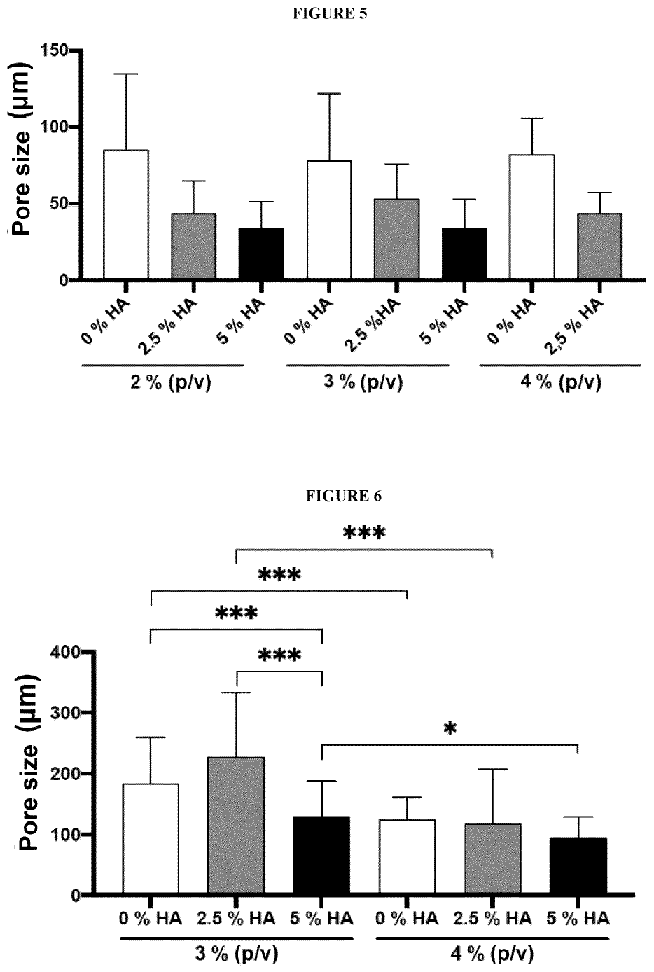

Next, the inventors evaluated the effect of final matrix concentration on pore size. They produced 2% (w/v), 3% (w/v) and 4% (w/v) matrices with different HA concentrations and analyzed their microstructure (FIG. 5).

The 3% (w/v) matrices tend to retain their structure depending on different HA concentrations, while the 2% (w/v) and 4% (w/v) matrices had powder-like structures, with a potentially reduced reproducibility. The SEM analysis confirmed the external porosity of the dehydrated matrices produced with a crosslinking time of 8 min, and demonstrated that the bulk density of the mesh increased in proportion to the matrix concentration. The 4% (w/v) matrices appear to comprise more pores but with smaller diameters, while the 2% (w/v) and 3% (w/v) matrices appear to comprise fewer pores but with larger dimensions. Pore size of dehydrated matrices with the same HA concentration did not vary as a function of final concentration. Again, the HA content seems to interfere with pore size in matrices with the same final concentration, but this trend does not appear to be statistically significant.

To confirm these results and assess internal porosity, the inventors quantified pore size in hydrated matrices, by cutting them down in the middle and producing histological slices by cryostat. FIG. 6 shows that in the 3% (w/v) matrices, the 5% HA matrices have smaller pore sizes than matrices comprising 0% and 2.5% HA. Furthermore, when comparing the 3% (w/v) and 4% (w/v) matrices, the 3% (w/v) matrices with 0% HA, 2.5% HA and 5% HA have significantly larger pores than the 4% (w/v) matrices with the same HA concentration. Importantly, in the 3% (w/v) matrices, there is no difference in pore size for matrices without HA and with 2.5% (w/v) HA, and the pore size is greater than 100 μm, which is the minimum pore size described in the literature to satisfy osteogenesis. From then on, work focused on 3% (w/v) matrices containing a maximum of 2.5% (w/v) HA.

Next, the inventors assessed the overall porosity of matrices without HA and 2.5% (w/v) HA after rehydration. They used the methodology described by Ma and Zang, based on overall densities of the fibrous matrix and skeletal density (Ma and Zhang, J. Biomed. Mater. Res. 1999, 46, 60). The skeletal density is taken as the density of polymers and HA. The matrices without HA and with 2.5% w/v HA did not differ in terms of porosity. The matrices without HA were 98.9±0.26% porous compared with 98.9±0.49% for 2.5% w/v HA. These results suggest that the presence of HA does not interfere with the overall porosity of hydrated matrices.

The retention of HA in the matrix structure was then studied by thermogravimetric analysis (TGA). The HA content retained in the matrix structure reached a plateau at 2.5% (w/v). The inventors therefore decided to use a maximum final HA concentration of 2.5% (w/v) for the rest of the tests, without however excluding the possibility of using matrices containing 1 or 2% HA given the results obtained at 2.5%.

The inventors then analyzed the HA distribution by EDX on the surface and inside the matrices. SEM images confirmed the surface and the internal porosity of matrices with or without HA. EDX analysis is an X-ray technique used to identify the elemental composition of materials; in this case the calcium content is visible as white dots. The experiments could show that hydroxyapatite is homogeneously distributed on the surface and in the matrices.

In parallel, biological evaluations were carried out to analyze cell survival and distribution in the matrices. Rat primary endothelial cells (EC)-RFP and mesenchymal stem cells derived from bone marrow (BMSC) were cocultured with the matrices and observed by confocal microscopy. The acquisitions made by the latter had a z volume of 130 to 190 μm and were analyzed using Imaris for 3D representation. After 7 days of co-culture, EC-RFPs were observed at the periphery and in the matrices, indicating that the matrices provide support for cell culture. To assess whether the cells colonize the matrix nucleus, they were cut down in the middle and then histological sections were prepared using a cryostat. The core of the samples was counterstained with DAPI (40,60-diamidino-2-phenylindole) and images were captured to detect RFP and DAPI, in order to assess cell colonization in the matrix core. For matrices without HA, no RFP or DAPI staining was observed in the core of the matrixes, but only at their periphery. On the other hand, RFP+ cells were homogeneously distributed within the matrices containing 2.5% HA.

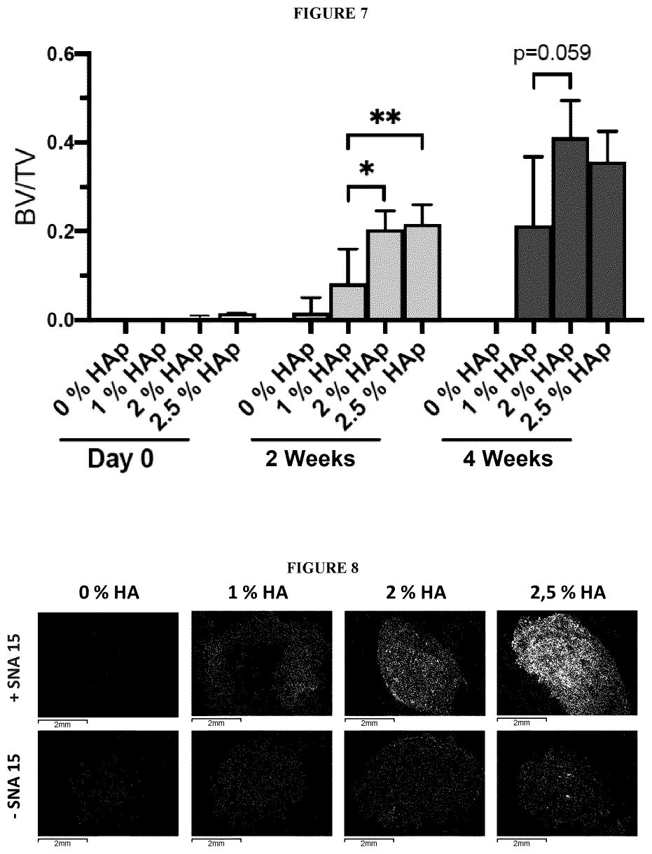

Following extensive matrix characterization, the inventors subcutaneously implanted mice with 3% w/v matrices HA-free or containing 1%, 2% and 2.5% w/v HA. The inventors then monitored the implantations on the day of implantation (Day 0), 2 (2 W) and 4 weeks (4 W) to assess the mineralization potential of the matrices using microCT. MicroCT confirmed the formation of ectopic mineralized tissue within the week 2 and week 4 implantations (FIG. 7). Once quantified, the volume of mineralized neotissue formed in matrices with 1%, 2% and 2.5% w/v HA increased over time. Matrices containing 2% and 2.5% w/v HA did not differ in the induction of mineralized neotissue formed after 2 or 4 weeks of implantation. Matrices with 2% and 2.5% produced a higher volume of mineralized tissue than the matrices with 1% w/v HA after 2 weeks. A p=0.059 was achieved when comparing the volume of mineralized tissue produced by matrices containing 1% and 2% w/v HA. Matrices without HA were not visible and did not produce significant mineralized tissue over time. A small amount of mineralized tissue was observed at 2 weeks in some animals, but was not detectable by week 4, presumably reabsorbed. These data suggest that matrices containing 1-2.5% HA have the potential for ectopic mineralization, and that 2% and 2.5% w/v HA perform best after 2 weeks.

In conclusion, the inventors have described a new matrix composite material cell-free and growth factor-free, able to induce formation of ectopic mineralized tissue in mice. This matrix is composed of ELPM80 and functionalized with biomimetic peptides responsible for recruiting nerve and vascular cells, stimulating cell colonization and retaining HA. Functionalized matrices combined with 1-2.5% (w/v) HA have osteoinductive properties.

### Example 2: Evidence of the Relevance of Peptide SNA15 in the Retention and Distribution of Hydroxyapatite Particles

The inventors produced a composite matrix, as described above, the organic part of which is composed of ELPM80-alkene, biomimetic peptides containing the adhesion sequences IKVAV, YIGSR, the proteolytic cleavage motif PVGLIG and also the calcium nucleation peptide SNA15, and the inorganic phase of which is composed of hydroxyapatite microparticles.

The calcium phosphate nucleation peptide SNA15, having the sequence DDDEEKFLRRIGRFG, is derived from the salivary protein statherin. A modified SNA15 peptide with sequence CβA-DDDEEKFLRRIGRFG-βAC was used.

Composite matrices containing or not this peptide SNA15 were produced with the same ELPs matrix concentration and by varying HA concentrations from 0%, to 2.5%.

An energy Dispersive X-Ray (EDX) analysis was carried out to determine the surface elemental composition of the materials, and more particularly the element calcium (identified as white dots in FIG. 8). It can be seen that matrices without HA contain little or no calcium. As expected, the more HA particles are added to the matrices, the more the signal increases. This phenomenon is even more visible when the gels contain the peptide SNA15.

A quantitative analysis shows that the atomic percentage of calcium in matrices containing SNA15 is greater than in those that do not: compared to matrices not containing the peptide SNA 15, there is 8 times more calcium in the 1% HA+SNA15 matrices, 8.9 times more in the 2% HA+SNA15 matrices and 12.6 times more in the 2.5% HA+SNA15 matrices.

It can be concluded that the peptide SNA 15 can substantially improve the retention of calcium phosphate particles, since there are between 8 and 12.6 more of them in its presence.

### Example 3: In Vivo Evaluation of ELP-Based Composite Matrices in Ectopic Sites and Bone Lesions

I. Ectopic Implantation of ELP Matrices Supplemented with Different Concentrations of Hydroxyapatite.

ELP and peptide matrices containing different concentrations of HA particles were implanted subcutaneously to select the one with the best mineralization, vascularization and innervation capacities. The four matrices differed only in their HA content (0%, 1%, 2% and 2.5% particles).

### I.1. Study of the Mineralization Potential of Composite Matrices

Mineralization of matrices implanted in subcutaneous sites was monitored by X-ray microtomography (micro-CT) as soon as the matrix was implanted (DO), and then after 15 and 30 days (D15 and D30) (FIG. 9). At day 0, no mineralization was observed in the 0%, 1% and 2% HA implants, whereas very little mineralization was observed in the 2.5% HA matrix (FIG. 8). These results indicate that HA particles at the concentrations studied are only slightly visible using the micro-CT technique.

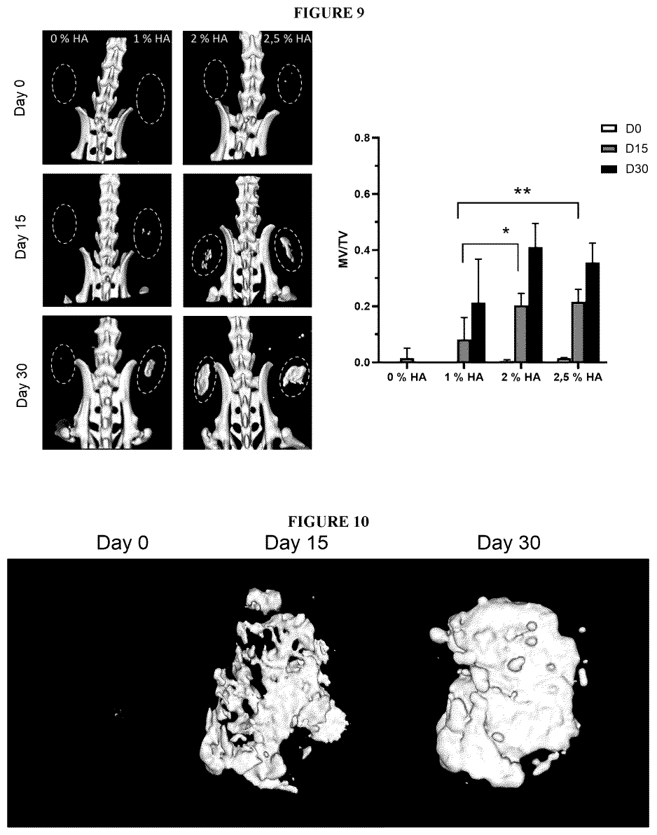

After 15 and 30 days of implantation, ectopic mineralized tissue was formed in the matrices containing HA particles. The volume of mineralized tissue formed in these composite matrices increased over time (FIG. 8). However, no mineralization was detected after implantation of the HA-free matrices, demonstrating the importance of these particles in the mineralization process. After 1 month of implantation (D30), matrices containing 1% HA induced an increase in mineral volume of 21±10%, 42±7% for those containing 2% HA, and 37±5% for matrices containing 2.5% particles. We did not measure any significant difference in the amount of mineralized tissue formed between matrices containing 2% and 2.5% HA after 30 days of implantation. The mineralization of the 2% HA matrix is shown in FIG. 10.

### 1.2. Study of the Osteoinductive Properties of Composite Matrices

After 4 weeks of implantation, the matrices were excised, embedded in paraffin and analyzed histologically using staining with Masson's trichrome to assess tissue architecture, cell colonization, inflammation and osteoid tissue formation.

Histological sections of the materials associated with the surrounding epidermal, adipose and muscular tissues show that after one month of implantation, very little fibrosis occurred around the ELPs matrices, whatever the formulation. This indicates that the matrices induce little inflammation and are not rejected. These results are in line with the biocompatibility demonstrated in vitro. On these sections we can see again a sheet-like structure and the presence of pores.

The presence of cell nuclei within the implanted matrices was also observed, indicating that cells had been able to penetrate these matrices.

As the tissues and matrices had not been decalcified, some areas of high mineralization were damaged during microtome cutting. However, the presence of mineralization in the form of dark purple crystals is noticeable. Osteoid tissue can also be seen at the periphery and within the matrices.

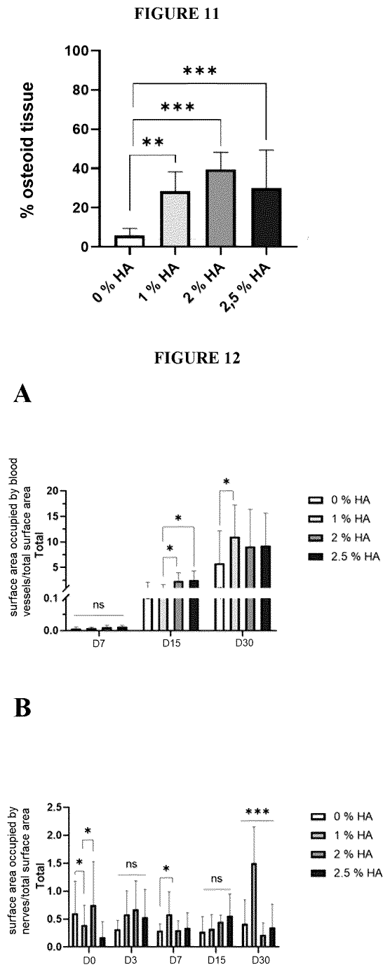

The amount of newly formed osteoid tissue (FIG. 11) increases significantly with the amount of HA in the matrix from 0 to 2%. By contrast, the values measured in matrices containing 2.5% mineral fraction were not significantly different from those containing 2% mineral fraction. Thus, the matrix containing 2% HA particles seems to be an advantageous formulation for stimulating a mineralization and osteoinduction process.

### 1.3. Study of the Angiogenic and Innervation Potential of ELP Matrices

The formation of vascular and nervous structures was observed by immunostaining for a vascular marker (Endomucin) and a neuronal marker (P III tubulin). During this study, histological sections of the matrices were taken at 0, 3, 7, 15 days and 1 month post-implantation to determine the kinetics of formation of these structures.

Regarding the formation of vascular structures at day 0, approximately 3 to 4 hours after implantation, no blood vessels were observed in the matrices. Three days after implantation, blood vessels appear mainly at the periphery of the matrices, then their infiltration is observed on day 7. After 15 days of implantation, vessels are present both at the periphery and inside the matrices. By day 30, the vascular network within the matrices became denser.

These observations are confirmed by quantification of blood vessels within the matrices studied (FIG. 1\acute{e}A). After 1 month of implantation, the vascular structures increase by around 10% in all matrices, whatever the HA concentration. Only a significant difference is observed between matrices without HA (0%) and those containing 1% HA.

The ability of the matrix to stimulate innervation was also demonstrated by the presence of nerves at the periphery of the material, particularly in the early stages after implantation. During bone regeneration, nerve structures are the first to appear, even in the early stages following bone damage.

This explains the presence of several nerve fibers visible at the periphery of the composite matrix from the 3rd day of implantation. After 1 month, nerve structures are still detected at the periphery of the matrix, but their diameter seems to have increased.

The quantitative results obtained by measuring the surface area occupied by nerve structures located at the periphery of the matrices in relation to the total surface area of the matrices confirmed the previous observations (FIG. 12B). In terms of formulation, it seems that composite matrices containing 1% HA significantly stimulate the formation of nerve structures after one month of implantation. This is particularly apparent when comparing these results with those obtained with matrices without HA or supplemented with 2.5% HA.

As the measured density of nerves or vessels can vary according to the location and/or angle of the tissue sections, we also used a technique that enables the entire tissue to be visualized in 3 dimensions after immunofluorescence labelling of the neuronal networks, without having to cut the sample. This technique allows to observe in 3 dimensions the autofluorescence of the matrix and the presence of networks of nerve structures expressing β III tubulin. In particular, a strong presence of nerve structures close to the matrix can be seen. Incidentally, the vascular networks seem to be homogeneously distributed.

1.4. Study of the Inflammatory Reaction after Subcutaneous Implantation of Composite Matrices.

The inflammatory reaction, when moderate, is an essential process for tissue regeneration and vascularization. On the other hand, too strong a response from the immune system can lead to rejection of the implant.

In the case of composite matrices, histological analysis using staining with Masson's trichrome (MT) did not reveal any inflammatory fibrotic capsules around the implants, whatever their composition.

To confirm these data, we carried out immunolabelling of immune cell populations. As inflammation markers, we used CD11b, which is expressed by the entire myeloid cell line (monocytes, macrophages, neutrophils) and CD45, which is a more specific marker for lymphocytes. We found that immune cells were recruited to the periphery of the matrix a few hours after implantation. These cells are detected up to day 7 post-implantation. Thereafter, after 3 and 6 months, the immune cells are only present inside the matrix. They could be macrophages that might play a role in the degradation of the matrix.

### 1.5. In Vivo Study of the Future of the Composite Matrix

To determine the lifespan of the matrices, subcutaneous implants were performed, and their future was monitored for up to 6 months after implantation.

Immediately after implantation (DO), the composite matrix containing 2% HA particles is clearly visible (reddish color), and blue HA crystals can be distinguished. After 1 month of implantation, the free spaces left by the pores filled in as a result of cell colonization. Several nuclei within the matrix can be identified. After 3 months of implantation, the structure of the matrix is no longer visible, which is probably due to its degradation by the immune system. The presence of a strong cellular component and of an extracellular matrix that fills the spaces previously occupied by the composite material can be noted. Similar results are observed after 6 months, but with an increase in mineralization of the entire area previously occupied by the composite matrix.

To conclude on these studies carried out at ectopic sites, the matrices developed from ELPs, peptides IKVAV/YIGSR and SNA15 revealed their ability to promote angiogenesis and innervation. Supplementing the matrices with HA gives them osteoinductive properties, as they are then able to form mineralized tissue and osteoid tissue.

In conclusion, as the composite matrix containing 2% HA performs best in terms of bone neoformation, neovascularization and generation of nerve structures, it was chosen for implants in bone sites.

II. Implantation of the Composite Matrix into Bone Site

The regenerative capacity of the composite matrix containing 2% HA was evaluated in rat models of femoral condylar defects and mandibular defects.

### II.1. Implantation in the Rat Femoral Condyle

Circular defects 3 mm in diameter were made in the femoral condyles of rats. Three conditions were tested: the unfilled defect corresponding to the negative control, the defect filled with our composite matrix, and the defect filled with the commercial Collapat® matrix used as a positive control.

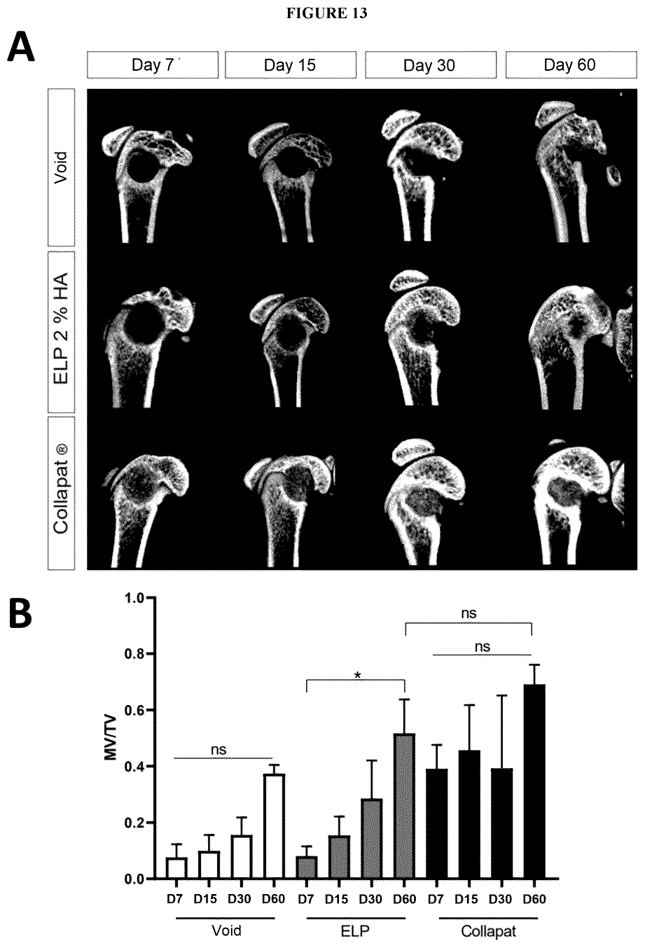

Micro-CT analyses were performed at different times after implantation (7, 15, 30 and 60 days) to monitor the mineralization of the condylar defects. 2D images of the femoral condyles reconstructed by 3D micro-CT are shown in FIG. 13A, and quantification of the volume of mineralized tissue relative to the total volume (MV/TV) is shown in FIG. 13B.

Micro-CT images reveal formation of a mineralized volume in lesions filled with composite matrices and Collapat®, while less mineralization is detected in unfilled defects (FIG. 13A).

The results of the quantitative analysis (FIG. 13B) show that there is partial mineralization of the unfilled defects with a non-significant increase in MV/TV as a function of time. At 7 days, this ratio is 8.6%±4.8%; it then increases to reach 10.3%±5.6% at 15 days, 14.7%±5.9% at 30 days and 37.5%±3.0% at 60 days. However, we observed site fractures in some animals, which complicated the quantification of MV/TV. Our previous work had shown that bone lesions 2.8 mm in diameter could repair naturally (Schlaubitz et al. 2014). In the present case, lesions with a diameter of 3 mm were made, probably weakening the lesion site and causing fractures that delayed or altered the repair kinetics. The micro-CT images nevertheless reveal mineralization at the periphery of the lesion, which explains the high MV/TV ratio of the area of interest (ROI) (FIG. 13B). In all cases, these images observed for unfilled lesions are different from those obtained after implantation of composite matrices.

Indeed, in this case a mineralization that begins at the inner edges of the lesion and increases with time post-implantation is observed. Initially, mineralization is essentially peripheral, then progresses over time towards the interior of the lesion. After 60 days of implantation, it can be seen that the cortical part has reformed, in contrast to the unfilled defect (FIG. 13A). Quantitative analyses show that MV/TV values increase significantly over time (FIG. 13B). They are 8.0±3.9% after 7 days of implantation, 15.6±7.0% after 15 days, 28.8±14.2% after 30 days and 56.0±6.9% after 60 days.

With the control material Collapat®, the MV/TV is 40.2±9.4% from day 7 of implantation, which is much higher than that measured with the ELP/peptides/HA composite matrix. This result was expected as this commercial material contains a high content of calcium phosphate particles, which induce an X-ray signal (FIG. 13A). No significant increase in mineralization from D7 to D30 was observed. As the MV/TV ratio varies little or not at all, the MV/TV ratio is 67.3±6.3% after 60 days.

Thus, if the three conditions studied are compared, it is observed that the quantity of mineralized tissue measured between days 7 and 60 is multiplied by 4.4 in the unfilled defects, by 7.1 with our composite matrix and by 1.7 in the case of Collapat®.

A histological analysis of the bone neoformation induced by the composite matrices was then performed.

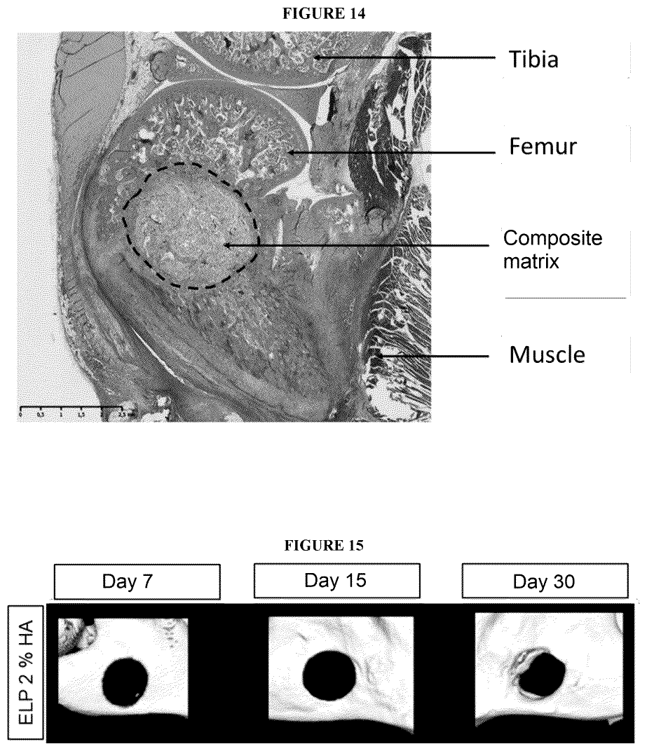

At 7 days, 15 days, 1 month and 2 months post-implantation, the femurs were explanted, decalcified and histological sections were taken. These sections were then stained with Masson's Trichrome (FIG. 14).

Histological sections of the lesion areas were observed. For unfilled lesions, the defect boundary was still clearly visible 7 days after surgery. After 15 days, the lesion was filled in with mainly collagenous tissue. By the thirtieth day, the borders of the lesion are difficult to distinguish, and organized bone-like tissue appears in the periphery.

The histological sections show that the ELP/peptide/HA composite matrices have integrated well into the bone defects. There is no visible demarcation between the lesion area and the material. The presence of numerous cell nuclei within the matrix confirms cellular colonization. In addition, the absence of fibrosis is a sign of the good biocompatibility of the matrices used. A quantification of newly formed osteoid tissue shows an increase from day 15, with the maximum amount measured at day 30. By two months, however, the amount of osteoid tissue had decreased. An analysis of the histological sections shows that bone formation occurs from the periphery towards the center of the defect, confirming the images observed by micro-CT (FIG. 13A).

Regarding the Collapat® material, a good osseointegration was also observed, as well as bone tissue formation from the periphery towards the center of the defect. A quantification of the newly formed osteoid tissue also showed a maximum of neoformation after 30 days, which is comparable to the kinetics observed with our composite matrices.

To specifically observe the presence of bone cells, an immunolabelling for osteocalcin, which is a specific marker for bone tissue, was performed. The images show that the unfilled lesions have few osteoblastic cells 7 and 15 days after the surgery. After 30 days, a few osteoblastic cells at the edge of the zone delimiting the bone lesion are detected.

In the case of composite matrices and Collapat®, osteocalcin is detected as early as day 15, and its presence is confirmed at days 30 and 60, confirming the presence of osteoblastic cells in the neoformed tissue.

To assess vascularization and innervation in condylar lesions, an analysis of vascular (Endomucin, Podocalyxin and Meca32) and neural (P III tubulin) markers using the whole-bone clearing technique was performed. This analysis made it possible to visualize the organization of the vascular and neuronal networks in the initially injured area. A multiple labelling was used to facilitate visualization of thick samples, as is the case for whole bone (Kirst et al. 2020, Cell 180, 780-795.e25).

2D sections of the lesion filled with the composite matrix containing 2% HA were analyzed after 30 days of implantation. The bone tissue appears green due to its autofluorescence, the vascular networks are cyan blue, and the neuronal extensions are red. 2D images obtained for each condition 15 and 30 days after implantation were also analyzed: unfilled lesions, lesions filled with composite matrices or with Collapat®.

In a lesion that had not been filled, a vascular network had developed. It seems to originate mainly from the peripheral muscular tissues. After 30 days, blood vessels and nerve structures within the area that had been injured are detected. However, histological analysis using TM staining showed that the injured area had been filled with soft, disorganized, collagen-rich tissue.

For lesions filled with composite matrices, the formation of a network of blood vessels throughout the matrix was observed as early as the fifteenth day.

In contrast, for defects filled with Collapat®, vascularization was mainly present at the outer periphery of the defect. Moreover, this vascularization was visible only from the 30th day after implantation. These qualitative results therefore show that the Collapat® matrix is less effective than our composite matrix in promoting vascularization of the implant.

As far as innervation is concerned, although the majority of fibers are present in the periosteum covering the cortical bone surface, a few fibers can be seen within the lesions for defects that have not been filled or have been filled with the composite matrix. By contrast, no nerve fibers were detected when the condylar defects had been filled with Collapat®.

### II. Implantation of the Composite Matrix in a Mandibular Defect

In order to evaluate the use of the material developed for craniofacial bone reconstruction, it was chosen to create a mandibular bone defect model. Among the bones of the skull, only two are mobile: the hyoid bone and the mandible. The mandible has the advantage of being a larger, easily accessible bone.

The mandibular defect also offers the advantage, compared with the calvaria defects classically used to test bone repair materials, of being subjected to high mechanical stresses. But these mechanical stresses are necessary for effective bone repair. At the mandible, the stresses are essentially linked to mastication. Therefore, this model of mandibular lesion was chosen to test bone repair materials, as it is better adapted to physiological reality than the calvarial defect.

To validate this model, a preliminary experiment was carried out. A circular defect 3 mm in diameter was created, and its repair was monitored using micro-CT. After 3 months, no bone repair was observed, indicating that the defect is indeed of critical size.

Preliminary micro-CT observations were made at different times after implantation (7, 15, 30 days) to monitor the mineralization of the mandibular defects. 3D images of the mandibles reconstructed by micro-CT filled with the composite matrix are shown in FIG. 15.

A partial reconstruction at the periphery of defect a is observed at day 30. The quantification of MV/TV was 0.037±0.010 on day 7 and 0.040±0.018 on day 15, i.e. comparable. On day 30, an increase in the MV/TV ratio (0.094±0.049) was measured.

Histological sections obtained after 15 days of implantation of the composite matrix were analyzed by TM staining.

Interestingly, a zone highlighting the presence of neoformed bone tissue with the presence of osteoblastic border cells, little boxes with osteocytes within a bone structure that seems to be lamellar, was observed at the junction of the bone and the matrix. Blood vessels were also seen in the neoformed tissue.

Furthermore, if one considers the reconstruction of the tissue from the periphery to the center of the lesion, it is important to note the tissue invasion which is accompanied by vascularization and also by formation of bone tissue in contact with the matrix, confirming their osteoconductive potential.

### Example 4: Conclusion on In Vivo Studies

In conclusion, various composite materials were tested in vivo in small animals at ectopic and orthotopic sites.

All the matrices tested integrated well with the surrounding tissues and did not induce any major inflammatory reaction. It was also shown that HA-containing matrices induced mineralization. Vascular structures invaded the implants, demonstrating their angiogenic potential. The ability of the matrices to stimulate innervation was also demonstrated by the presence of nerves at the periphery of the material, particularly in the early stages following implantation.

The matrix containing 2% HA was selected for its ability to promote mineralization and for its angiogenic potential. It was implanted in condylar and mandibular bone defects.

Bone reconstruction was improved after implantation of composite matrices in a condylar defect compared with an unfilled defect. Organized bone structures expressing osteocalcin were identified in these hydrogels. An organized vascular network developed throughout the bone lesion, which was not the case for the Collapat® control material.

The results obtained in the mandibular defect model show very satisfactory results for the osteoconductive and angiogenic properties of the composite matrices. Histological analysis showed that the materials were well integrated. Bone and vascular structures were found as early as 15 days after implantation.

These results therefore demonstrate the therapeutic value of composite matrices according to the invention.

1-14. (canceled)

15. A composite material comprising:
at least one elastin-like peptide,
at least one bioactive peptide,
one peptide able to induce calcium phosphate nucleation; and
calcium phosphate.

16. The composite material according to claim 15, wherein the elastin-like peptide comprises at least one alkenylated residue before formation of the composite material.

17. The composite material according to claim 15, wherein the elastin-like peptide is of formula MW[VPGVGVPGMG(VPGVG)₂]ₓ (SEQ ID NO: 43) in which x is an integer between 15 and 25 and the methionine residues are alkenylated before formation of the composite material.

18. The composite material according to claim 15, wherein the elastin-type peptide is the peptide ELPM80 of formula MW[(VPGVG)(VPGMaG)(VPGVG)₂]₂₀ (SEQ ID NO: 1) where Ma represents the alkenylated methionine residue of formula (III): 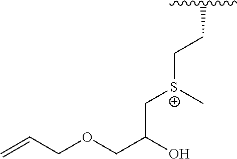
before formation of the composite material.

19. The composite material according to claim 15, wherein said at least one bioactive peptide is an adhesion peptide able to recruit cells of interest.

20. The composite material according to claim 15, wherein said at least one bioactive peptide is an adhesion peptide of formula IKVAV-GGG-PVGLIG-GGG-YIGSR (SEQ ID NO: 19).

21. The composite material according to claim 15, wherein the peptide able to induce calcium phosphate nucleation is a peptide of sequence DSSEEKFLRRIGRFG (SEQ ID NO: 31).

22. The composite material according to claim 15, wherein the peptide able to induce calcium phosphate nucleation is a peptide of sequence DDDEEKFLRRIGRFG (SEQ ID NO: 47).

23. The composite material according to claim 15, wherein the calcium phosphate corresponds to hydroxyapatite.

24. The composite material according to claim 23, comprising between 1 and 3% (w/v) hydroxyapatite.

25. The composite material of claim 23, comprising 2% (w/v) hydroxyapatite.

26. A method of promoting innervation, osteogenesis and/or angiogenesis comprising implanting the composite material according to claim 15 into a subject in need of innervartion, osteogenesis and/or osteogenesis.

27. A method of controlling bone development comprising implanting the composite material according to claim 15 into a subject in need of bone development.

28. A method of repairing bone comprising implanting the composite material according to claim 15 into bone in need of repair.


SEQUENCE LISTING (47 sequences)

SEQ ID NO: 1 - protein, 17 aa, synthetic construct
  REGION 1..17: note = ELPM80
  REPEAT 3..17: note = 20 repetitions
  REPEAT 13..17: note = 2 repetitions
MWVPGVGVPGMGVPGVG

SEQ ID NO: 2 - protein, 4 aa, synthetic construct
  REGION 1..4: note = Peptide d'adhesion
REDV

SEQ ID NO: 3 - protein, 6 aa, synthetic construct
  REGION 1..6: note = Peptide d'adhesion
GRGDSP

SEQ ID NO: 4 - protein, 5 aa, synthetic construct
  REGION 1..5: note = Peptide d'adhesion
IKLLI

SEQ ID NO: 5 - protein, 5 aa, synthetic construct
  REGION 1..5: note = Peptide d'adhesion
IKVAV

SEQ ID NO: 6 - protein, 5 aa, synthetic construct
  REGION 1..5: note = Peptide d'adhesion
PDSGR

SEQ ID NO: 7 - protein, 5 aa, synthetic construct
  REGION 1..5: note = Peptide d'adhesion
YIGSR

SEQ ID NO: 8 - protein, 4 aa, synthetic construct
  REGION 1..4: note = Peptide d'adhesion
DGEA

SEQ ID NO: 9 - protein, 10 aa, synthetic construct
  REGION 1..10: note = Peptide d'adhesion
RNIAEIIKDI

SEQ ID NO: 10 - protein, 9 aa, synthetic construct
  REGION 1..9: note = Peptide d'adhesion
  SITE 2: note = MISC_FEATURE - bAla
  SITE 8: note = MISC_FEATURE - bAla
CAIKVAVAC

SEQ ID NO: 11 - protein, 9 aa, synthetic construct
  REGION 1..9: note = Peptide d'adhesion
  SITE 2: note = MISC_FEATURE - bAla
  SITE 8: note = MISC_FEATURE - bAla
CAYIGSRAC

SEQ ID NO: 12 - protein, 6 aa, synthetic construct
  REGION 1..6: note = Peptide cible
PVGLIG

SEQ ID NO: 13 - protein, 7 aa, synthetic construct
  REGION 1..7: note = Peptide cible
QPQGLAK

SEQ ID NO: 14 - protein, 9 aa, synthetic construct
  REGION 1..9: note = Peptide cible
GPLGLSLGK

SEQ ID NO: 15 - protein, 8 aa, synthetic construct
  REGION 1..8: note = Peptide cible
GPLGMHGK

SEQ ID NO: 16 - protein, 8 aa, synthetic construct
  REGION 1..8: note = Peptide cible
GPQGIAGQ

SEQ ID NO: 17 - protein, 8 aa, synthetic construct
  REGION 1..8: note = Peptide cible
GPQGIWGQ

SEQ ID NO: 18 - protein, 8 aa, synthetic construct
  REGION 1..8: note = Peptide cible
VPMSMRGG

SEQ ID NO: 19 - protein, 22 aa, synthetic construct
  REGION 1..22: note = Peptide d'adhesion
IKVAVGGGPVGLIGGGGYIGSR

SEQ ID NO: 20 - protein, 22 aa, synthetic construct
  REGION 1..22: note = Peptide d'adhesion
YIGSRGGGPVGLIGGGGIKVAV

SEQ ID NO: 21 - protein, 22 aa, synthetic construct
  REGION 1..22: note = Peptide d'adhesion
PVGLIGGGGYIGSRGGGIKVAV

SEQ ID NO: 22 - protein, 22 aa, synthetic construct
  REGION 1..22: note = Peptide d'adhesion
PVGLIGGGGIKVAVGGGYIGSR

SEQ ID NO: 23 - protein, 22 aa, synthetic construct
  REGION 1..22: note = Peptide d'adhesion
IKVAVGGGYIGSRGGGPVGLIG

SEQ ID NO: 24 - protein, 21 aa, synthetic construct
  REGION 1..21: note = Peptide d'adhesion
YIGSRGGGIKVAVGGGPVGLI

SEQ ID NO: 25 - protein, 26 aa, synthetic construct
  REGION 1..26: note = Peptide d'adhesion
  SITE 2: note = MISC_FEATURE - bAla
  SITE 25: note = MISC_FEATURE - bAla
CAIKVAVGGGPVGLIGGGGYIGSRAC

SEQ ID NO: 26 - protein, 26 aa, synthetic construct
  REGION 1..26: note = Peptide d'adhesion
  SITE 2: note = MISC_FEATURE - bAla
  SITE 25: note = MISC_FEATURE - bAla
CAYIGSRGGGPVGLIGGGGIKVAVAC

SEQ ID NO: 27 - protein, 26 aa, synthetic construct
  REGION 1..26: note = Peptide d'adhesion
  SITE 2: note = MISC_FEATURE - bAla
  SITE 25: note = MISC_FEATURE - bAla
CAPVGLIGGGGYIGSRGGGIKVAVAC

SEQ ID NO: 28 - protein, 26 aa, synthetic construct
  REGION 1..26: note = Peptide d'adhesion
  SITE 2: note = MISC_FEATURE - bAla
  SITE 25: note = MISC_FEATURE - bAla
CAPVGLIGGGGIKVAVGGGYIGSRAC

SEQ ID NO: 29 - protein, 26 aa, synthetic construct
  REGION 1..26: note = Peptide d'adhesion
  SITE 2: note = MISC_FEATURE - bAla
  SITE 25: note = MISC_FEATURE - bAla
CAIKVAVGGGYIGSRGGGPVGLIGAC

SEQ ID NO: 30 - protein, 26 aa, synthetic construct
  REGION 1..26: note = Peptide d'adhesion
  SITE 2: note = MISC_FEATURE - bAla
  SITE 25: note = MISC_FEATURE - bAla
CAYIGSRGGGIKVAVGGGPVGLIGAC

SEQ ID NO: 31 - protein, 15 aa, synthetic construct
  REGION 1..15: note = Peptide de nucleation
DSSEEKFLRRIGRFG

SEQ ID NO: 32 - protein, 10 aa, synthetic construct
  REGION 1..10: note = Peptide de nucleation
EFLRRIGRFG

SEQ ID NO: 33 - protein, 5 aa, synthetic construct
  REGION 1..5: note = Peptide de nucleation
DHTKE

SEQ ID NO: 34 - protein, 9 aa, synthetic construct
  REGION 1..9: note = Peptide de nucleation
SSSEEIVPN

SEQ ID NO: 35 - protein, 12 aa, synthetic construct
  REGION 1..12: note = Peptide de nucleation
DEGEQPRPFPFP

SEQ ID NO: 36 - protein, 7 aa, synthetic construct
  REGION 1..7: note = Peptide de nucleation
WEWLHYW

SEQ ID NO: 37 - protein, 11 aa, synthetic construct
  REGION 1..11: note = Peptide de nucleation
DGDDGEAGKIG

SEQ ID NO: 38 - protein, 10 aa, synthetic construct
  REGION 1..10: note = Peptide de nucleation
GPAGPHGPPG

SEQ ID NO: 39 - protein, 15 aa, synthetic construct
  REGION 1..15: note = Peptide de nucleation
VLSGGTTMYASLYAG

SEQ ID NO: 40 - protein, 8 aa, synthetic construct
  REGION 1..8: note = Peptide de nucleation
VLGYIQIR

SEQ ID NO: 41 - protein, 13 aa, synthetic construct
  REGION 1..13: note = Peptide de nucleation
DNLPNPEDNKNYQ

SEQ ID NO: 42 - protein, 19 aa, synthetic construct
  REGION 1..19: note = Peptide de nucleation
  SITE 2: note = MISC_FEATURE - bAla
  SITE 18: note = MISC_FEATURE - bAla
CADDDEEKFLRRIGRFGAC

SEQ ID NO: 43 - protein, 17 aa, synthetic construct
  REGION 1..17: note = ELPM40
  REPEAT 3..17: note = 15 repetitions
  REPEAT 13..17: note = 2 repetitions
MWVPGVGVPGMGVPGVG

SEQ ID NO: 44 - protein, 15 aa, synthetic construct
  REGION 1..15: note = Peptide
  REPEAT 1..15: note = 5 repetitions
  REPEAT 11..15: note = 2 repetitions
VPGVGVPGMGVPGVG

SEQ ID NO: 45 - protein, 5 aa, synthetic construct
  REGION 1..5: note = Peptide scramble IKVAV
VKAIV

SEQ ID NO: 46 - protein, 5 aa, synthetic construct
  REGION 1..5: note = Peptide scramble YIGSR
GYSRI

SEQ ID NO: 47 - protein, 15 aa, synthetic construct
  REGION 1..15: note = Peptide d'adhesion
DDDEEKFLRRIGRFG